(19) United States
(12) Patent Application Publication
(10) Pub. No.: US 2003/0104006 A1
(43) Pub. Date: Jun. 5, 2003

(54) Hyaluronidase activity and allergenic cell activity inhibitor

(76) Inventors: Takaaki Maekawa, Inashiki-gun (JP); Hiroko Isoda, Tsukuba-shi (JP)

Correspondence Address: WENDEROTH, LIND & PONACK, L.L.P., 2033 K STREET N. W., SUITE 800, WASHINGTON, DC, 20006-1021, US

(21) Appl. No.: 10/239,747

(22) PCT Filed: Mar. 21, 2001
(86) PCT No.: PCT/JP01/02236

(30) Foreign Application Priority Data
May 9, 2000 (JP) 2000-136283

(51) Int. Cl. A61K 35/84; C12N 1/16

(52) U.S. Cl. 424/195.15; 435/254.1

(57) ABSTRACT

Disclosed is a novel hyaluronidase activity and allergenic cell activity inhibitive substance useful as an effective ingredient in an anti-inflammatory or antiallergic medicament, which is an ethyl alcohol extract from body tissues or mycelia tissues of a fungus Agaricus blazei. The desired inhibitive activity of the ethyl alcohol extract is significantly higher than the corresponding hot water extract of the same fungous tissues. This difference is very prominent when the Agaricus blazei fungus cultured in a culture medium containing wheat bran and pinewood chips in combination is used as the starting material of the ethyl alcohol extraction.

### TECHNICAL FIELD

The present invention relates to a novel hyaluronidase activity and allergenic cell activity inhibitor and a method for the preparation thereof as well as medical applications thereof as an effective ingredient of anti-inflammatory and antiallergic medicaments.

### BACKGROUND ART

It is a noticeable trend in recent years not only in Japan but also in many other countries throughout the world that allergic diseases are increasing year by year with an estimated rate of 10% annual increase in the number of allergic patients. So to say, allergic diseases belong to a kind of civilization diseases and are considered to be caused by uptake of heterogeneous proteins as a consequence of the changes in the dietary and in the housing conditions. In addition, the increase in the allergic patients in recent years is partly due to the manifestation of latent patients as is the case in relation to the pollinosis which has come to the public attention.

As is known, hyaluronic acid is a biopolymer compound playing a very important role in the processes of ontogeny and homeostasis of tissues as well as phlogogenic reactions and wound healing. On the other hand, hyaluronidase is an enzyme to promote hydrolysis of hyaluronic acid and contained in large amounts in the connective tissues of mammals. It is established that hyaluronidase has an activity to cause inflammation in the human body so that inflammation could be alleviated by inhibiting the activity of hyaluronidase. This is the reason for the recent demand for development of an effective hyaluronidase activity inhibitor as an effective ingredient of anti-inflammatories.

On the other hand, hyaluronic acid is known as a natural moisturizing factor (NMF) and formulated in various kinds of toiletry and cosmetic preparations such as milky lotions and creams so that a hyaluronidase activity inhibitor could have an application also as an ingredient of toiletry and cosmetic preparations to prevent decomposition of hyaluronic acid.

It is also known that allergenic cells such as the basophilic leukocytes among the blood cells and mast cells in the tissues isolate enzymes such as β-hexosaminidase and histamine by degranulation to induce instantaneous allergic reactions such as asthma, hay fever and atopic eczema. Accordingly, it would be a good assumption that allergic diseases could be alleviated by inhibiting degranulation of basophilic leukocytes so that an effective inhibitor against the degranulation activity of basophilic leukocytes has an application as an antiallergic agent.

A basidiomycetous fungus called agaric and having a scientific name of Agaricus blazei is an edible fungus as a kind of health foods having certain medicinal activity and widely employed from old. While a great variety of constituents are contained in the body of an agaric fungus of which, however, little information is available, it is reported that a water-soluble compound contained in the body of an agaric fungus is identified to be β-D-glucans, in particular, β-(1,3)-D-glucan and β-(1,6)-D-glucan, and has an anti-cancer activity.

### DISCLOSURE OF INVENTION

In view of the above described various situations, the present invention has an object, on one hand, to provide a novel hyaluronidase activity and allergenic cell activity inhibitor and an efficient method for the preparation thereof as well as to propose, on the other hand, a novel application of the inhibitor as an effective ingredient in anti-inflammatory and antiallergic medicaments.

After extensive investigations undertaken with an object to develop a novel material capable of inhibiting the hyaluronidase activity and allergenic cell activity, the inventors have arrived at a discovery that an ethyl alcohol extract from tissues of bodies or mycelia of a basidio-mycetous fungus or, in particular, an agaric fungus exhibits strong inhibition against the hyaluronidase activity and allergenic cell activity and that the extract can be used as an effective ingredient in anti-inflammatory and antiallergic medicaments leading to completion of the present invention on the base of this discovery.

Thus, the present invention provides a hyaluronidase activity and allergenic cell activity inhibitor which is an ethyl alcohol extract from tissues of bodies or mycelia of Agaricus blazei or, generally, a basidiomycetous fungus. The origin of the agaric fungus, of which the fungous body tissues or mycelia tissues are subjected to extraction with ethyl alcohol, is not particularly limitative including the fungous bodies grown in the wild, cultured fungous bodies and cultured bodies of mycelia. The gathering seasons, growth ages, culturing conditions and culturing periods of the agaric fungus bodies are also not particularly limitative.

### BEST MODE FOR CARRYING OUT THE INVENTION

Although the desired activity of the ethyl alcohol extract from agaric fungous bodies or mycelia is not uniform depending on the growing conditions such as the formulation of the culture medium, any fungous bodies grown by a variety of growing methods can be used as the base material in the present invention. While a variety of species are included in the genus of Agaricus, those agaric fungi can be used either singly or as a combination of two kinds or more as the base material in the invention. It is of course that the desired activity of the ethyl alcohol extract can be synergistically increased by using several kinds of different agaricous fungi each giving high activity in combination. Combined use of a first agaric fungus giving high hyaluronidase activity inhibitive effect and a second agaric fungus giving high inhibitive effect against degranulation activity of basophilic leukocytes could lead to exhibition of the anti-inflammatory and antiallergic activity for a composite medicament.

According to the method of the invention, the hyaluronidase activity and allergenic cell activity inhibitor can be obtained as an ethyl alcohol extract from tissues of bodies or mycelia of an agaric fungus. The ethyl alcohol as the extractant of the fungous tissues is not particularly limited to absolute alcohol but can contain water and other impurities each in a limited amount provided that the impurities do not disturb extraction of the active matters from the fungous tissues. When the ethyl alcohol as the extractant contains water, the content of water therein should not exceed 40% by weight or, desirably, 20% by weight. The volume of the ethyl alcohol used in the extraction treatment is not particularly limitative but should be sufficient to completely dissolve out the water-insoluble active matters contained in the fungous tissues.

When culturing of the agaric fungus is conducted for obtaining the fungous bodies or mycelia, the culture medium is admixed with 0.2 to 3 g/liter of a milled flour of agricultural and forestry raw materials such as wheat bran, bagasse, pinewood chips, pine seeds, wheat, foxtail millet, rice, rice straws, wheat straws and the like either singly or as a combination of two kinds or more. The culturing is conducted as a tank culture process usually for 3 to 14 days.

The fungous bodies or mycelia as grown and separated from the liquid culture medium are thoroughly washed with water to remove the adhering liquid culture medium and foreign materials followed by chopping into pieces, an ultrasonic treatment and grinding by using a grinding machine such as homogenizers into a pulpy form. It is optional that this grinding process is preceded by air-drying or freeze-drying so as to give a dry powder by grinding.

In the next place, the fungous bodies or mycelia tissues thus ground are admixed with an appropriate volume of ethyl alcohol and the mixture is agitated at a temperature up to the boiling point of ethyl alcohol to dissolve out the alcohol-soluble matter in the fungous bodies or mycelia tissues followed by separation of the extract solution from the insoluble matter. Preferably, this extraction treatment is repeated several times in order to completely extract the extractable matter.

The thus obtained ethyl alcohol extract solution contains the hyaluronidase activity and allergenic cell activity inhibitor. The extract can be used as such as the effective ingredient of an anti-inflammatory or antiallergic medicament, if necessary, after dilution or concentration. A powdery material can be obtained by subjecting the extract solution to spray drying or freeze-drying. The thus obtained dry powder exhibits strong hyaluronidase activity and allergenic cell activity inhibition.

Being a constituent in the tissues of bodies or mycelia an agaric fungus which is employed as a food from old, the ethyl alcohol extract prepared in the above-described manner has absolutely no safety problem against human body. Accordingly, the ethyl alcohol extract can be used as an additive to toiletry and cosmetic preparations, medicaments and foodstuff either alone or in combination with other additives. The adding amount of the agaric extract naturally depends on the specific purpose of its addition to exhibit the desired effect. As an additive to a toiletry or cosmetic preparation, for example, the adding amount thereof can be in the range from 2 to 20 ppm by weight calculated as solid. When used as an additive in an orally administrable medicament, the administration dose thereof is usually in the range from 0.2 to 2 mg/kg body weight per day calculated as, solid, if necessary, in portions a day.

The medicaments to which the agaric extract is added can be in a variety of medicament forms depending on the object of administration and administrative route including tablets, capsules, injection fluids, instillation fluids, triturations, suppositories, granules, ointments, suspensions, emulsions and others.

Since the ethyl alcohol extract from tissues of bodies or mycelia of agaric fungus exhibits strong anti-inflammatory and antiallergic activity, a health food having anti-inflammatory or antiallergic activity can be prepared by adding the extract to a conventional food provided that the food is not inhibitive against the anti-inflammatory or antiallergic activity of the extract. Types of the foods and beverages suitable to this purpose include bakery foods such as breads, confectioneries such as candies, various kinds of precooked foods, seasonings and so on without particular limitations.

In the following, the present invention is described in more detail by way of examples.

Following is the base formulation of the culture media used in the culturing of the agaric fungus conducted in the Examples.

| | | |
|---|---|---|
| Magnesium sulfate heptahydrate | 0.5 | g |
| Iron(II) sulfate heptahydrate | 0.01 | g |
| Yeast extract | 1.0 | g |
| Barley flour | 1.0 | g |
| Sucrose | 30 | g |
| Deionized water | 1000 | ml |

### EXAMPLES

Eight culturing runs A, B, C, D, E, F, G and H of Agaricus blazei were carried out at 28 to 29° C. each in a liquid culture medium of the above described base formulation with or without addition of an auxiliary additive or additives selected from wheat bran, bagasse, which served as the sources of nitrogen, phosphorus and potassium, and pinewood chips each in an amount of 1 g. The culture medium was inoculated with 5 g of the fungous bodies calculated as wet. The auxiliary additives and the culturing time in these culturing runs were as shown in Table 1 below.

After the end of the culturing time as indicated, a 10 g portion of wet bodies of the fungus as grown was taken from the culture medium and thoroughly rinsed with water to remove the liquid culture medium adhering to the surface of the fungous bodies. Thereafter, the fungous bodies were mashed by 5 cycles of ultrasonic treatments at 10 second intervals at 20° C. with a 100 watt output and 50 kHz ultrasonic frequency. The fungous body mash was admixed with four times by weight of absolute ethyl alcohol and thoroughly agitated therein at room temperature. The thus obtained dispersion of the fungous body mash was subjected to centrifugal separation at 1500 rpm for 2 minutes to collect the supernatant as an ethyl alcohol extract.

The ethyl alcohol extract solution was subjected to spray drying to give about 100 mg of an extract powder. For comparison, the same extraction procedure as above was undertaken for the fungous bodies obtained in the culturing runs A to H excepting for the replacement of four times by weight of ethyl alcohol as the extractant with four times by volume of hot water to give a hot water extract after 15 minutes of boiling of the aqueous dispersion of the fungous body mash.

The above obtained ethyl alcohol extract solutions and hot water extract solutions were each subjected to the evaluation tests of the hyaluronidase activity inhibition and allergenic cell activity inhibition in the following assay procedures 1 and 2, respectively.

Assay 1

A 0.025 ml portion of a solution prepared by dissolving 2.83 mg of bovine testis-origin hyaluronidase (a product by Sigma Co.) in 1 ml of a 0.1M acetate buffer solution of pH 4.0 was admixed with 0.2 ml of a solution prepared by dissolving sodium chloride in a concentration of 0.3M in a 0.1M acetate buffer solution of pH 4.0 and the mixture was kept at 37° C. for 20 minutes. The mixture was further admixed with 0.1 ml of the above obtained ethyl alcohol extract solution or hot water extract solution as dissolved in a 1/15M phosphate buffer solution of pH 7.0 containing potassium dihydrogen phosphate and disodium hydrogen phosphate and the mixture was incubated at 37° C. for 20 minutes. The mixture was then further admixed with 0.2 ml of a solution prepared by dissolving 1.83 mg of a potassium salt of hyaluronic acid originating in cock's comb (a product by Sigma Co.) in 1 ml of a 1M acetate buffer solution and kept standing at 37° C. for 20 minutes. Thereafter, the mixture was further admixed with 0.1 ml of a 0.4N aqueous sodium hydroxide solution and 0.1 ml of a 0.8M aqueous sodium borate. solution and boiled for 3 minutes followed by cooling and addition of 3.0 ml of a solution prepared by dissolving 10 g of 4-dimethylaminobenzaldehyde and 12.5 ml of 10N hydrochloric acid in 87.5 ml of acetic acid and 10 times diluted with acetic acid. After standing for 20 minutes at 37° C., this solution was subjected to the spectrophotometric measurement of the absorbance at a wavelength of 585 nm.

The hyaluronidase activity inhibition HI was calculated from the equation:

HI(%)=[(Y−X)−(Z−X)]/(Y−X)×100,

in which X is the absorbance of the control solution containing neither the hyaluronidase nor sample solution, Y is the absorbance of the solution containing hyaluronidase only and Z is the absorbance of the solution for measurement prepared by adding hyaluronidase to the sample solution. The results for the culturing runs A to H are shown in Table 1.

TABLE 1

| | |
|---|---|
| HI, % | IHₑₓ, % |

| | | Culturing | Hot | Ethyl | Hot | Ethyl |
|---|---|---|---|---|---|---|
| Culture | Auxiliary | time, | water | alcohol | water | alcohol |
| run | additives | hours | extract | extract | extract | extract |
| A | None | 72 | 15.6 | 22.1 | 0.6 | 36.4 |
| B | None | 120 | 12.3 | 19.8 | 0 | 26.9 |
| C | Wheat | 72 | 8.7 | 23.7 | 5.8 | 47.8 |
| | bran | | | | | |
| D | Bagasse | 72 | 9.4 | 24.6 | 26.8 | 85.2 |
| E | Wheat | 72 | 12.6 | 18.6 | 17.4 | 74.3 |
| | bran | | | | | |
| | Bagasse | | | | | |
| F | Wheat | 120 | 13.4 | 21.2 | 0 | 38.9 |
| | bran | | | | | |
| | Bagasse | | | | | |
| G | Wheat | 72 | 11.4 | 40.6 | 34.8 | 92.4 |
| | bran | | | | | |
| | Pinewood | | | | | |
| | chips | | | | | |
| H | Wheat | 120 | 18.6 | 86.8 | 5.3 | 64.7 |
| | bran | | | | | |
| | Pinewood | | | | | |
| | chips | | | | | |

As is clear from this table, the HI value for the ethyl alcohol extract is definitely greater than the HI value of the hot water extract for the same agaric fungous bodies grown in each of the culturing runs A to H. The difference is particularly prominent when culturing is conducted in a liquid culture medium admixed with wheat bran and pinewood chips in combination as is the case in the culturing runs G and H.

Assay 2

Each of the ethyl alcohol extracts and hot water extracts was subjected to the assay of allergenic cell activity inhibition in the following manner by using basophilic leukocytes as the allergenic cells. In conducting the assay tests, following preparatory solutions were prepared beforehand.

(1) Substrate Solution, 5 mM Concentration

The solution was prepared by dissolving 85.5 mg of 4-nitrophenyl-N-acetyl-β-D-glucosamide (Sigma N9376) as the substrate in 50 ml of a 50 mM citrate buffer solution of pH 4.5.

(2) Mixed Releasing Solution, pH 7.7

The solution was prepared by dissolving, in 500 ml of water, 3.42 g of sodium chloride, 201 mg of potassium chloride, 98.6 mg of magnesium sulfate heptahydrate, 504 mg of glucose, 3.0 g of a buffering agent (HEPES), 111 mg of calcium chloride and 0.5 g of bovine serum albumin followed by adjustment of the pH to 7.7 and sterilizing filtration.

(3) Reaction Stopper Solution

The solution was a 100 mM carbonate buffer solution of pH 10.0 containing 1.06 g/100 ml of sodium carbonate and 0.84 g/100 ml of sodium hydrogencarbonate.

(4) 4 μg/ml DNP-BSA Solution

The solution was prepared by 25 times dilution of a solution containing 1 mg/10 ml of bovine serum albumin (DNP-BSA, Cosmobio LG0017) in a phosphate buffer solution with the same phosphate buffer solution followed by sterilizing filtration.

(5) 1 mg/ml Solution of Mouse Anti-DNP IgE Antibody (Sigma D8406)

(6) 3mM Ketotifen Solution

The solution was prepared by dissolving 12.8 mg of ketotifen fumarate having a molecular weight of 425.5 (Sigma K2628) in 10 ml of a phosphate buffer solution followed by sterilizing filtration.

The assay procedure was as described below by using a 75 cm² flask which could serve for inoculation of two or more of 24-well plates.

After a trypsin treatment of the cells in a flask to facilitate releasing of the cells from the wall by keeping the flask in a carbon dioxide incubater for 5 minutes with addition of trypsin followed by gentle tapping of the flask, the liquid culture medium was added to the flask followed by a centrifugal treatment. The thus trypsin-treated cells were suspended in about 5 ml of the culture medium and the cell concentration of the suspension was adjusted to 5×10⁵ cells per ml by dilution with the culture medium followed by the addition of the preparatory solution (5) in a 1/3333 volume to give a final concentration of 0.3 μg/ml with shaking to effect thorough suspension of the cells. A 24-well plate was inoculated with the cell suspension in a volume of 500 μl per well and kept standing in a carbon dioxide incubater overnight to sensitize the cells.

The cells after the overnight standing in the incubater were twice washed with a 1 ml volume of a phosphate buffer solution and then 260 μl of the preparatory solution (2) were added thereto in combination with 20 μl of the sample solution or the phosphate buffer solution as a control followed by 10 minutes incubation in the carbon dioxide incubater. After further incubation for 1 hour in the carbon dioxide incubater with addition of 20 μl of the preparatory solution (4), the solution was recovered in an Eppendorf tube and subjected to a centrifugal treatment. A 100 μl portion of the supernatant was admixed with 400 μl of the preparatory solution (1) and incubated at 37° C. for 30 minutes followed by the addition of 1 ml of the preparatory solution (3) to be subjected to the spectrophotometric measurement at a wavelength of 405 nm.

Measurements were made for three samples (n=3) in which a 1300 μl portion of the preparatory solution (2) to be added to the cells was admixed in advance with 100 μl of the sample solution. The volume of the sample solution was decreased to 10 μl when the sample solution was a 70% ethyl alcohol extract solution in order to reduce the influence by the ethyl alcohol. The solution left after addition to the cells was admixed likewise with the preparatory solutions (4) and (3) for the measurements of the absorbance to give the background level of the spectrophotometric measurement.

Inhibition against hexosaminidase liberation IHₑₓ, %, was calculated by using the equation:

IHₑₓ(%)=[1−(a−b)/(c−d)]×100,

in which a is the absorbance with addition of the sample to the cells, b is the absorbance with addition of the sample in the absence of the cells, c is the absorbance as a control by the admixture of the cells with a phosphate buffer solution or 70% ethyl alcohol 5-times diluted with a phosphate buffer solution and d is the absorbance by the addition of the control in the absence of the cells. The results of the calculation for the values of IHₑₓ in each of the culture runs A to H are shown also in Table 1.

As is understood from the IHₑₓ values tabulated in Table 1, the ethyl alcohol extract of the fungous body tissues in each of the culture runs exhibits stronger allergenic cell activity inhibition than the corresponding hot water extract. In particular, the difference is prominent in the culture runs with addition of wheat bran and pinewood chips in combination to the culture medium as is the case in the culturing run G.

Industrial Applicability

The above described experimental results support the conclusion that the ethyl alcohol extract from tissues of bodies and/or mycelia of the agaric fungous contains substances capable of exhibiting inhibition against hyaluronidase activity and allergenic cell activity in large amounts as compared with the hot water extract so that the extract is useful as an effective ingredient in an anti-inflammatory and antiallergic medicament. Since the ethyl alcohol extract is free from the safety problem against human body and can be prepared by a simple and inexpensive procedure, the applicability of the extract is not limited to the medicaments mentioned above but also includes the application as an additive in foods and cosmetic or toiletry compositions in general.

1. A hyaluronidase activity and allergenic cell activity inhibitive substance which is an ethyl alcohol extract from body tissues or mycelia tissues of a fungus Agaricus blazei.

2. A method for the preparation of a hyaluronidase activity and allergenic cell activity inhibitive substance which comprises the steps of:
(a) mashing body tissues or mycelia tissues of a fungus Agaricus blazei to give a mash of the fungus tissues;
(b) adding ethyl alcohol to the mash of the fungous tissues to give an ethyl alcohol extract solution; and
(c) removing at least a part of the ethyl alcohol from the ethyl alcohol extract solution.

3. The method for the preparation of a hyaluronidase activity and allergenic cell activity inhibitive substance according to claim 2 wherein the fungous bodies or mycelia of the fungus are obtained by culturing the fungus in an artificial culture medium.

4. The method for the preparation of a hyaluronidase activity and allergenic cell activity inhibitive substance according to claim 3 wherein the artificial culture medium contains wheat bran and pinewood chips in combination each in an amount in the range from 0.2 to 3 g per liter.

5. A method for therapeutic anti-inflammatory treatment of a patient which comprises administrating the patient with the hyaluronidase activity and allergenic cell activity inhibitive substance as defined in claim 1.

6. A method for therapeutic antiallergic treatment of a patient which comprises administrating the patient with the hyaluronidase activity and allergenic cell activity inhibitive substance as defined in claim 1.
